(19) United States
(12) Patent Application Publication
(10) Pub. No.: US 2010/0145187 A1
(43) Pub. Date: Jun. 10, 2010

(54) Catheter

(76) Inventors: Joerg Weber, Edling (DE); Bernd Beck, Ragendingen (DE)

Correspondence Address: GLENN PATENT GROUP, 3475 EDISON WAY, SUITE L, MENLO PARK, CA, 94025, US

(21) Appl. No.: 12/513,340

(22) PCT Filed: Oct. 31, 2007
(86) PCT No.: PCT/EP2007/009458

§ 371 Date: Feb. 1, 2010

(30) Foreign Application Priority Data
Nov. 3, 2006 (DE) 10 2006 051 978.7

(51) Int. Cl. A61M 25/00; A61B 8/00; B23P 17/04; B26D 7/27

(52) U.S. Cl. 600/424; 604/523; 29/428; 53/396

(57) ABSTRACT

So that a catheter can be made visible in ultrasound images easily and with relatively little effort, and hence positional control of a catheter can be enabled with relatively little effort, an ultrasound marking recognizable in the body of a living being by means of ultrasound is provided at the distal end of a catheter, wherein the catheter has a lumen, and the ultrasound marking is arranged in the lumen near the distal end such that it is removable via a proximal end of the catheter.

### CROSS-REFERENCE TO RELATED APPLICATIONS

This application is a U.S. national entry of PCT Patent Application Serial No. PCT/EP2007/09458 filed 31 Oct. 2007, and claims priority to German Patent Application No. 102006051978.7 filed on 3 Nov. 2006, which is incorporated herein by reference in its entirety.

### BACKGROUND OF THE INVENTION

The present invention relates to catheters and introducing the same in general, and to venous catheters in embodiments and central venous catheters in particular, such as in the form of multi-lumen catheters, for example.

A venous catheter, or also a central venous catheter, designates a flexible tube with one or more channels and/or lumens therein, which is introduced into the venous system until its distal end is short of the heart, and so that it is possible to take measurements through the lumen openings, such as of the central venous pressure, and/or to supply liquid, such as electrolyte and nutrient solutions or medicine. A single-lumen catheter only possesses one lumen, the opening of which is at the distal end of the catheter, for example. In multi-lumen catheters the lumen openings may be arranged in a spaced manner along the length of the catheter so as to prevent the reagents administered via the lumens from interfering with each other. The introduction or catheterization of the venous catheter is mostly done via a central vein by way of the Seldinger technique, wherein at first a guidewire is placed at the target position via a needle. Via the guidewire, the catheter is advanced and guided up to a desired position so as to end there. The navigation and/or the positional control of the wire may, for example, take place via an electrocardiographic lead (ECG lead) and by utilizing the electrical impulses triggering the heartbeats, and particularly utilizing the polarization reversal in the case of the guidewire gliding past the sinus node. Once the guidewire has reached the desired position, the venous catheter is advanced via the guidewire until its proximal end corresponds to a corresponding mark on the guidewire, whereupon the guidewire is removed again. As an alternative to or in addition to the ECG positional control of the venous catheter, the positional control may take place by means of X-ray, to which end the material of the flexible venous catheter is constituted of barium sulfate, for example, to a great extent.

A relatively great effort regarding navigation or positional control of the distal end of the venous catheter is a disadvantage of the previously outlined procedure, because the ECG lead does not always provide reliable results or may only be employed to a limited extent, and the X-ray control is connected with high costs and much time involved.

### SUMMARY

According to an embodiment, a catheter may have an ultrasound marking, which is recognizable by means of ultrasound in the body of a living being, at a distal end thereof, wherein the catheter may have a lumen, and the ultrasound marking is arranged in the lumen near the distal end such that it is removable via a proximal end of the catheter, wherein the ultrasound marking is a tube closed at its distal end, which is introduced into the lumen.

According to another embodiment, a catheter system may have a catheter with a lumen; and an ultrasound marking insertable into the lumen of the catheter, which is recognizable in the body of a living being by means of ultrasound and is elongated so as to be able to extend from near a distal end of the catheter through the lumen beyond a proximal end of the catheter to a connection of the catheter so as to project from the connection, wherein the ultrasound marking is a tube closed at its distal end, which is insertable into the lumen.

According to another embodiment, a method of preparing a catheter for introducing the same may have the step of: inserting an ultrasound marking, which is recognizable in the body of a living being by means of ultrasound and is elongated so as to be able to extend from near a distal end of the catheter through the lumen beyond a proximal end of the catheter to a connection of the catheter so as to project from the connection, into the lumen to the proximity of the distal end, wherein the ultrasound marking is a tube closed at its distal end.

It is a central idea of the present invention that, with relatively little effort, a catheter can easily be made visible in ultrasound images, and hence positional control of a catheter can be enabled with relatively little effort, by providing an ultrasound marking means, which is recognizable in the body of a living being by means of ultrasound, at the distal end of a catheter, for example temporarily, so as to be removed again after having successfully guided the distal end of the catheter to a target position of the ultrasound marking means.

According to one embodiment, it is provided to introduce a passive or active elongated probe into a lumen of a catheter for temporary use during the catheter navigation or positional control and the ensuing removal and to package the same in this form as a catheter system, for example, and to sterilize the same.

### BRIEF DESCRIPTION OF THE DRAWINGS

Embodiments of the present invention will be detailed subsequently referring to the appended drawings, in which:

FIG. 1 is a schematic drawing of a two-lumen catheter; and

FIG. 2 is a schematic sectional view of the distal end of the two-lumen catheter of FIG. 1, according to a comparative example;

FIG. 3 is a schematic drawing of a sectional view of the distal end of the two-lumen catheter of FIG. 1, according to a further comparative example;

FIG. 4 is a sectional view of the two-lumen catheter of FIG. 2 and/or FIG. 3 along the sectional line A-A;

FIG. 5 is a schematic sectional view of a distal end of a single-lumen catheter with a passive probe of a catheter system introduced, according to an embodiment of the present invention; and

FIG. 6 is a schematic drawing of the proximal end of the arrangement of FIG. 5.

### DETAILED DESCRIPTION OF THE INVENTION

Before explaining embodiments of the present invention in greater detail in the following with reference to the drawings, it is pointed out that like elements are provided with like reference numerals in these figures, and that repeated description of these elements will be omitted.

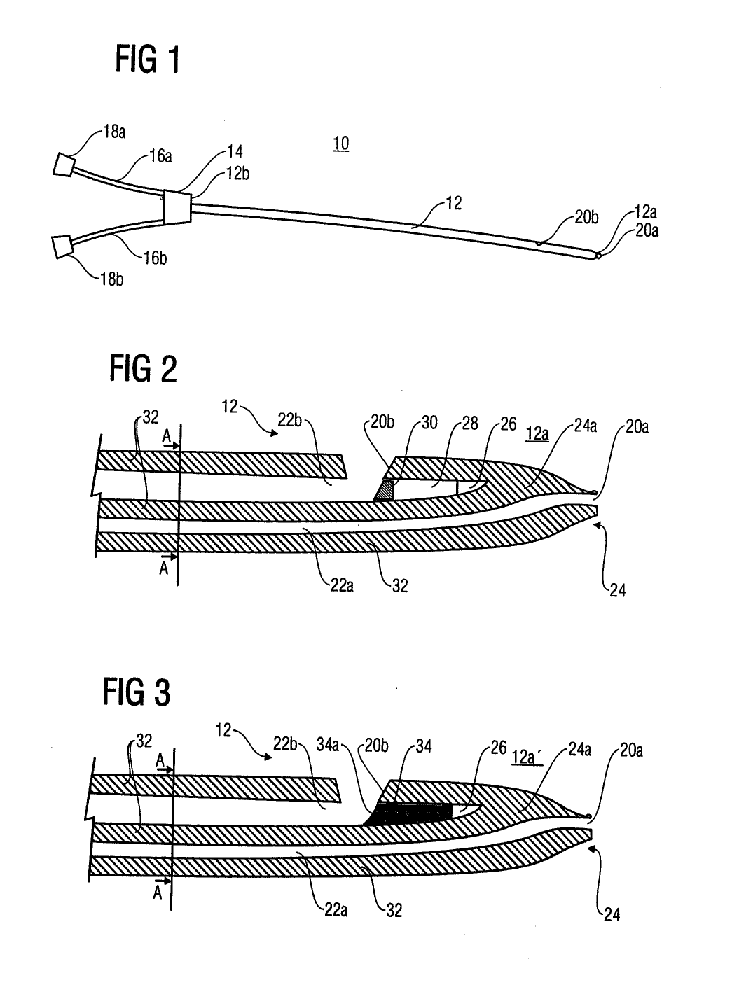

FIG. 1 exemplarily shows a two-lumen catheter which may be employed as a venous catheter, for example. The catheter of FIG. 1, generally shown at 10, includes a flexile catheter tube 12 with a distal end 12a and a proximal end 12b, a distributor or manifold 14, two connecting tubes 16a and 16b, and two plug connections 18 and 18b, which may be formed as screwed connectors and are also sometimes referred to as Luer-Lock connections in the following. The catheter tube 12 comprises, in its interior (not visible in FIG. 1), two channels or lumens, one of which opens at a lumen opening 20a arranged at the distal tip, and the other lumen opening at a lateral opening 20b arranged at a distance from the distal tip or more proximally, but still near the distal end 12a. The distributor 14 connects one of the lumens to the connecting tube 16a and the other lumen to the connecting tube 16b, which in turn are connected to one of the Luer-Lock connections 18a, 18b each, at the proximal end 12b, so that further tubes can be connected thereto for the supply of liquid or taking blood or other fluidic coupling to the lumen openings 20a, 20b.

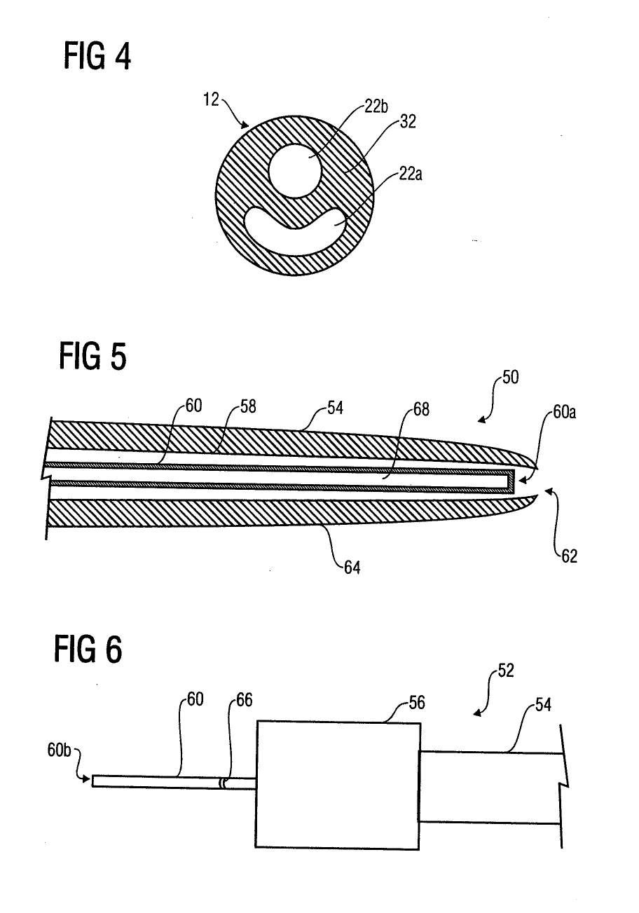

FIG. 2 shows the distal end 12a of the tube 12 in greater detail, for a comparative example of the present invention. What can be seen are the two lumens 22a and 22b of the catheter tube 12a, which extend therealong and, as shown in FIG. 4, have different cross-sections. Alternatively, they may, of course, also have the same cross-section. As can be seen, the lumen 22b opens at the lateral lumen opening 20b, which is arranged slightly distant from the distal tip 24 of the catheter, whereas the lumen 22a opens at the lumen opening 20a at the distal tip 24. However, the lumen 22b extends slightly beyond the lumen opening 20b in the direction of the distal tip 24, so as to end at the distal tip 24 at a part 24a of the tip 24. The lumen 22b thus forms, from the lumen opening 22b in the direction of the distal tip 24, a sack opening, which may optionally be filled partially or completely with material 26 at its distal end, as will still be described in the following in connection with the description of manufacture. However, this sack opening further forms a cavity 28 closed by a closing element 30 at the open end of the sack opening and/or near the lumen opening 20b. The closing element 30 closes the cavity 28 in a fluid-tight manner in the part of the lumen 22b that is distal with respect to the lumen opening 20b, and for example consists of the same material as the material 32 forming the lumens 22a and 22b. For example, the cavity 28 is filled with air, but may also be filled with a vacuum or a foamed or porous material.

The two-lumen catheter according to FIGS. 2 and 4 offers advantages when used as a venous catheter, for example. So as to illustrate this, introducing the catheter will be described in the following. At first, the multi-lumen catheter is introduced into the patient, such as into a vein of the neck. After the distal end 12a has been introduced into the vein, the catheter 10 is advanced further along the vein in the direction of the heart, wherein this may be done with or without using a guidewire. In the patient's body, the two-lumen catheter 10 is observed by means of ultrasound. Although the material 32 of the catheter cannot be visualized very well in the ultrasound images due to the low acoustic impedance difference to the surrounding blood, the cavity 28 located near the distal tip 24 certainly can, which cavity reflects the ultrasound waves of an ultrasound transducer well because of its interface from the acoustically thick material 32 or material 32 of high acoustic impedance to the interior of the cavity 28, which is acoustically thin and/or has lower impedance, thus leading to high contrast in the images. Depending on the observation by means of ultrasound and/or the location of the cavity 28 in the ultrasound images, the catheter may then be navigated and/or advanced to the desired position, for example such that the tip 24 is positioned immediately before the heart. Advantageously, ultrasound devices mean less effort, bulk and cost and are less complex in their handling than X-ray devices, so that the positional control, when introducing the catheter according to the FIGS. 2 and 4, is simplified as compared with an X-ray solution.

Before a further example of a two-lumen catheter is described with reference to FIG. 3, one possible manufacturing method for the catheter of FIG. 2 will be described in the following. At first, a catheter tube with the lumens 22a and 22b is produced from the material 32, such as by extruding. The result is a tube with a continuously constant cross-section, such as with that of FIG. 4. Then, the lumen 22 is closed at the distal end 12a, so that the lumen 22a still has its lumen opening 20a at the tip of the catheter tube at the distal end 12a, however. To this end, for example, the lumen 22b is closed with a material 26 at the distal sectional edge of the extruded tube, and a tip 24 with the closing part 24a is attached to the sectional area thus prepared. The material 26 and the material of the tip 24 may each be equal to or different from the material 32 of the catheter tube. The material 32 is, for example, polyurethane with or without a barium sulfate proportion. Closing the lumen 22b may also be performed via thermal closing and a thermal and/or mechanical shaping of the tip 24 to the shape shown in FIG. 2. Prior to or following the formation of the tip 24 and/or distal-side closure of the lumen 22b, the lateral lumen opening 20b is formed, such as by a cutting process taking place in a transverse direction. Then, the lumen 22b is closed by means of the closing element 30, wherein glue, which is brought to the desired position through the opening 20b, is used as the closing material 30, for example. Alternatively, the same material as the material 32 or material 26 may be used for the element 30, which is suitably connected to the material 32, such as by means of a solvent or an adhesive. The atmosphere present when closing the cavity with the element 30, such as air, then is in the cavity 28.

As an alternative, it is, of course, also possible to introduce the closing element 30 from the distal side through the lumen 22b and/or the cavity 28, which still is open at the distal end at this time, into the lumen 22b prior to closing the lumen 22b and/or the formation of the distal tip 24, and then finally close the cavity 28 only later when forming the tip 24. The formation of the lumen opening 20b could also take place in another order with respect to the remaining steps. Furthermore, prior to closing the cavity 28 by the closing element 30 and/or the part 24a of the tip 24, it could be filled with a foamed or porous material 34. Due to its air and/or gas inclusions, this material also leads to a good contrast in ultrasound images. This foamed material would then be separated from the surrounding serum, such as blood, by the closing element 30 and/or the material 32, so that its possibly rugged surface does not offer any point of attack for biological growth or fouling and/or the attachment of germs, and the surface of the catheter may still be continuously smooth to prevent attachments of such germs. As an alternative, the closing material may be omitted, and the porous material 34 may be produced with a smooth interface, an example of which will be explained in the following with reference to FIG. 3.

FIG. 3 shows an example alternative to FIG. 2 of a distal end 12a′ of the two-lumen catheter of FIG. 1, but with the cross-section A-A from FIG. 4 also applying to this example. The comparative example of FIG. 3 differs from that of FIG. 2 in that the portion of the lumen 22b with the lateral lumen opening 20b, which extends from the opening 20b to the distal end of this lumen 22b, is not closed in a cavity, but is only filled with foamed material 34 having inclusions, which again have the above-outlined advantages in the ultrasound imaging. For example, polyurethane with or without suitable additives may be used as the foamed material 34. Similarly, as described previously with respect to the closing element 30, the foamed material 34 may be introduced through the lateral lumen opening 20b when producing the catheter of FIG. 3 sometime after forming the opening 20, and advantageously also the attachment of the tip, or through a distal opening of the lumen 22b at a time at which the tip 24 with the closing part 24a has not yet been attached and/or formed. With respect to the advantages of the catheter of FIG. 3 when introducing same, reference is made to the statements regarding the catheter of FIG. 2, which also apply to the comparative example in this respect. Regarding the manufacture of the catheter of FIG. 3, the above statements with respect to FIG. 2 apply, unless described in a deviating manner with respect to FIG. 3.

Although part of the rough surface of the foamed material (34) is exposed to the surrounding serum, such as blood, in the catheter of FIG. 3, the risk of germ contamination or attachments is very low, since the zone is very limited locally. After all, the material 34 may also be produced with a smooth interface 34a.

Finally, it is to be mentioned that the above comparative examples have dealt with a two-lumen catheter only exemplarily, and that they are also applicable to multi-lumen catheters with more lumens. In general, in an n-lumen catheter with n−1 lateral lumen openings or lateral eyes, the portion of each of these n−1 lumens arranged between the respective lateral eye and the end of the lumen directed to the distal end of the multi-lumen catheter may be used to form a closed cavity 28 and/or accommodate foamed material 34. Furthermore, it is pointed out that the cross-sections visible in FIG. 4 also only are of exemplary nature. Thus, the outer perimeter of the cross-section of the catheter tube 12 may also be shaped in a different way than circular, as shown in FIG. 4. As previously mentioned, the material 26 further may be missing at the end of the lumen 22b.

In the foregoing, it has been described that the material possesses gas bubbles. Other materials are also possible, however, namely ones mixed with ultrasound-visible substances. This applies both to the material 34 in FIG. 3 and to the “filling” of the cavity 28 in FIG. 2. In the present application, for example, something is to be considered recognizable or visible in the body of a living being by means of ultrasound if it actively sends out ultrasound or reflects ultrasound impinging through the inside of the body with a sufficient reflection coefficient, such as by using a material with an acoustic impedance of, for example, less than 10% of the acoustic impedance of water or less than 10% of the acoustic impedance of the catheter material, in the interior of the catheter.

Moreover, the above comparative examples may be modified in that, instead of providing a “passive” ultrasound marking in the form of the cavity 28 and/or the material 34, an active ultrasound marking means is provided at the distal end of the multi-lumen catheter, or at least near the lumen openings. For example, in the part of the lumen 22b between the lumen opening 20b and the proximal end thereof, an active “probe” in the form of an ultrasound-emitting transmitter may be inserted. Advantageously, this active probe has its own energy supply in the form of a battery, for example. The active probe could merely be put into the lumen 22b, advantageously only up to the proximity of the lateral eye 20b, or even all the way to the sack opening of the lumen 22b.

Instead of an active probe generating or emitting ultrasound signals, however, also a “passive” probe in the form of a gas-filled ball or a gas-filled tube, which is closed at the front and the rear, could be inserted and/or mounted in the lumen 22b, such as up to the opening 22b or up to the cavity 28 and/or the sack opening from FIG. 2 and from FIG. 3, respectively.

The insertion of the passive or active probe, and/or the manufacture of a multi-lumen catheter with such a probe, may take place in the prescribed way by inserting the probe into the cavity 28 in the manufacture according to FIG. 2, for example, before the same is closed by the material 30 or the distal tip 24, or by replacing the material introduction step for the material 34 in the manufacture according to FIG. 3 by a probe introduction step. The advantages resulting by way of the modified comparative examples just described with an active and passive probe in connection with introducing the catheter are easily obvious from the description of the examples of FIGS. 2 and 3, which also applies to the modifications in this respect, even though, when using an active probe, the advantage does not lie in the utilization of the high reflectivity at the interface from acoustically thick to acoustically thin material, but in the utilization of the ultrasound signal directly emitted by the active probe. A suitable active probe may comprise means for activating and maybe deactivating the ultrasound signal generation, depending on a triggering event, such as depending on an electromagnetic trigger signal, so that the ultrasound signal generation can be limited to the time period of the employment of the catheter.

The preceding comparative examples dealt with catheters in which the ultrasound marking means in form of the cavity 28, the material 34 and/or the passive/active probe has been provided to permanently remain in the multi-lumen catheter, and particularly in the otherwise unused part of a lumen with a lateral eye, even though, in the case of the merely inserted passive/active probe, the same basically could be removed via the lumen 22b. In the following embodiment, the introduction of a catheter accurately to a target position, such as short of the heart, is facilitated by the fact that a lumen of a catheter, which may also be a single-lumen catheter in this embodiment, is temporarily provided with a passive or active probe that may be removed again through the lumen. Such an embodiment will be explained in greater detail with reference to FIGS. 5 and 6, which show the distal end 50 and/or the proximal end 52 of a catheter 54, at the proximal end 52 of which a connection 56 is arranged, such as a Luer-Lock connection. According to the embodiment of FIGS. 5 and 6, the catheter 54 is a single-lumen catheter with a lumen 58 and forms, together with a passive probe 60, which is also shown, a catheter system facilitating the introduction of the catheter 54, as described in the following. To this end, the passive probe 60 here exemplarily is formed as a flexible tube, with the outer perimeter of its cross-section being small enough for the tube 60 to be introduced into the lumen 58 via the connection 56 or the proximal end 52. In particular, the tube 60 is long enough so as to extend from the distal end 50 through the lumen 58 beyond the connection 56 to the outside. More specifically, the tube 60 is long enough so as to extend from a proximity of a lumen opening 62 of the lumen 60 at a lumen tip 64 of the distal end 50 through the lumen 58 beyond the connection 56, wherein a marking 66 is provided visibly on the exterior of the tube 60 for an operator, so as to be at a predetermined distance to the connection 56, such as immediately in front of it, at a position of the tube 60 as inserted up to the proximity of the tip 62. The tube 60 is hollow on the inside and further is closed at its distal end 50a and also at its proximal end 60b, for example, so as to form a gas-filled space 68 on the inside, for example.

After the components of the catheter system of FIGS. 5 and 6 have been described in the foregoing, its use when introducing the catheter 54 will be described in the following. Prior to the use and/or the introduction of the catheter 54, or during the use and/or the introduction, the catheter 54 and the flexible tube 60 are, for example, in the state shown in FIGS. 5 and 6, namely in the state of the tube 60 being introduced with its distal end 60a up to the proximity of the distal tip 62 of the catheter 54 into the lumen 58, and packaged in a sterile way, for example. Once the catheter 54 has been introduced into a vein or catheterized, for example, it is advanced along the vein to the proximity of a target position, such as before the heart. Since the tube 60 is in the interior of the lumen 58 as a passive probe and extends up to the proximity of the tip of the lumen 62, the position of the catheter tip 62 and/or the entire catheter 54 relative to the target position can be controlled by means of ultrasound, as described previously, and thus the navigation of the catheter 54 with its tip 62 can be guided to the target position, since good contrast of the passive probe 60 results from the interface of the tube material to the tube interior 68 in the ultrasound images. On the basis of the marking 66, the physician here can ensure that the distal end 60a is in the desired proximity to the catheter tip 62. The remaining offset, as can be seen in FIG. 5, for example is known to the operator in advance, so that he or she may take same into account in the positional control during the observation of the ultrasound images. After having guided the catheter tip 62 to the target position in this manner, as desired, the passive probe 60 can be removed therefrom through the lumen 58, to which end the tube 60 can be drawn at its projecting proximal end. The passive probe 60 here is provided as a disposable article, for example.

For preparing the introduction of the catheter 54, the tube 60 thus has been introduced into the lumen 58 up to the distal end 50, so as to then be packaged and sterilized together with the catheter 54, for example, such as by a packaging permeable for sterilization gas.

With reference to the previous embodiment of FIGS. 5 and 6, it is pointed out that the same is not limited to a single-lumen catheter. Rather, a passive probe 60 can, of course, also be employed in a multi-lumen catheter, wherein the introduction may take place into each of the lumens, such as, but not exclusively the lumen with its lumen opening at the catheter tip. Furthermore, it pointed out that, instead of the passive probe described, also an elongated active probe could be used, which, for example, has a similar exterior as the previously described flexible tube 60, but has an ultrasound transmitter at its distal end. Finally, also a solid flexible rod may be used as a passive probe, as long as it enables ultrasound visibility, or a flexible rod of foamed material or the like.

Although it has previously been described that the passive probe 60 is packaged together with the catheter 54 in an introduced state, for example, it may also be provided for both components of the catheter system, namely the catheter and the passive probe 60, to be packaged separately. When introducing the catheter 54, it may at first be introduced into a vein, for example, and advanced a little, so as to then introduce the passive probe 60 into the lumen 58. Either the passive probe 60 may be advanced therethrough through the vein to the target position, in order to let the catheter 54 follow only when the distal end 60a of the passive probe reaches the target position as controlled by ultrasound, and then remove the passive probe 60 from the lumen 58 like in the foregoing, or the catheter is navigated to its target position via ultrasound control with the aid of the probe.

Finally, it is to be pointed out that it is possible to integrate, into the existing software of an ultrasound device, a software routine acting as a means for displaying a signal for catheter navigation, and capable of recognizing the ultrasound marking means, such as the closed cavity and/or the foamed material filling, or the passive/active probe in the ultrasound images of the ultrasound image sequence generated while one of the previously described catheters is pushed along the vein in the direction of the heart, and also recognize the location thereof, and display a signal for the physician, for example, on the basis of this recognized location, serving for navigation of the multi-lumen catheter to the desired position before the heart. For example, an acoustic sound is given off at the moment at which the catheter tip glides past the desired location, wherein the software routine to this end also takes into account the longitudinal offset between the ultrasound marking means, such as the cavity (28) and/or the foamed material (34) or the active/passive probe observed in the ultrasound images, to the tip (24), for example. Of course, the navigation signal display routine just described may also be implemented in firmware or in hardware. Moreover, the routine also may recognize the target position automatically, for example by the user only indicating to the routine that “the distal end” is to be arranged “before the venous entry of the heart” and/or a “central venous catheter” is desired, whereupon the routine automatically recognizes this location from certain features in the ultrasound images.

Finally, it is still to be pointed out that the present invention is not limited to venous catheters. Rather, the present invention may also be applied to other catheters and to other catheter applications in serums other than blood.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. A catheter with an ultrasound marking, which is recognizable by means of ultrasound in the body of a living being, at a distal end thereof, wherein the catheter comprises a lumen, and the ultrasound marking is arranged in the lumen near the distal end such that it is removable via a proximal end of the catheter, wherein the ultrasound marking is a tube closed at its distal end, which is introduced into the lumen.

2. The catheter according to claim 1, wherein the ultrasound marking is located so as to be able to extend from near the distal end through the lumen beyond the proximal end to a connection of the catheter so as to project from the connection.

3. The catheter according to claim 1, wherein the ultrasound marking comprises an ultrasound signal transmitter for generating and outputting an ultrasound signal.

4. The catheter according to claim 3, wherein the catheter is formed as a catheter comprising a first lumen with a lumen opening at a tip of the catheter at the distal end, and a second lumen with a lateral lumen opening arranged more proximally than the first lumen opening, wherein the ultrasound marking is arranged in a part of the second lumen located between the first lumen opening and the second lumen opening.

5. The catheter according to claim 1, which is suitable as a central venous catheter.

6. A catheter system, comprising
a catheter with a lumen; and
an ultrasound marking insertable into the lumen of the catheter, which is recognizable in the body of a living being by means of ultrasound and is elongated so as to be able to extend from near a distal end of the catheter through the lumen beyond a proximal end of the catheter to a connection of the catheter so as to project from the connection, wherein the ultrasound marking is a tube closed at its distal end, which is insertable into the lumen.

7. A method of preparing a catheter for introducing the same, comprising:
inserting an ultrasound marking, which is recognizable in the body of a living being by means of ultrasound and is elongated so as to be able to extend from near a distal end of the catheter through the lumen beyond a proximal end of the catheter to a connection of the catheter so as to project from the connection, into the lumen to the proximity of the distal end, wherein the ultrasound marking is a tube closed at its distal end.

8. The method according to claim 7, further comprising:
packaging the catheter, with the ultrasound marking inserted, in a package.
